(19) United States
(12) Patent Application Publication
(10) Pub. No.: US 2017/0080034 A1
(43) Pub. Date: Mar. 23, 2017

(54) LACTOBACILLUS COMPOSITION ALLOWING THE STIMULATION OF HUMAN AND ANIMAL JUVENILE GROWTH IN CASES OF MALNUTRITION

(71) Applicants: ECOLE NORMALE SUPERIEURE DE LYON, LYON (FR); Université Claude Bernard Lyon 1, VILLEURBANNE (FR); Centre national de la recherche scientifique, PARIS (FR)

(72) Inventors: François LEULIER, SEREZIN DU RHONE (FR); Gilles STORELLI, SALT LAKE CITY, UT (US); Martin SCHWARZER, NOVY JICIN (CZ); Maria Elena MARTINO, LYON (FR)

(21) Appl. No.: 15/311,457

(22) PCT Filed: May 15, 2015
(86) PCT No.: PCT/EP2015/060753

§ 371 Date: Nov. 15, 2016

(30) Foreign Application Priority Data
May 16, 2014 (FR) 1454422

(51) Int. Cl. A61K 35/747; A23L 33/00; G01N 33/74; A23L 33/135; C12R 1/25; A61K 49/00

(52) U.S. Cl. CPC A61K 35/747; C12R 1/25; A61K 49/0008; G01N 33/74; A23L 33/135; A23L 33/30; A61K 2035/115

(57) ABSTRACT

The invention relates to a pharmaceutical or probiotic composition comprising at least one Lactobacillus strain with intestinal tropism, especially selected from the species Lactobacillus plantarum, Lactobacillus fermentum and Lactobacillus casei, used to stimulated juvenile growth in cases of malnutrition especially characterised by a protein deficiency. The strains can be selected from a vinegar fly model and/or a mouse model. The invention also relates to a method for probiotic treatment using said composition.

The present invention relates to a pharmaceutical or probiotic composition giving the possibility of promoting human and animal juvenile growth in the case of malnutrition. This composition comprises as active principle or ingredient at least one bacterium with intestinal tropism, preferably a lactic bacterium.

Described as <<an additional unit>>, the intestinal microbial community (or intestinal microbiota) plays a key role beneficial for the host by exerting many biological functions, such as contributing to the efficiency of digestion, metabolism of the substrates, control against pathogens, or further setting into place and homeostasis of immune responses. Imbalances between different intestinal bacterial populations have sometimes deleterious repercussions leading to the development of diverse pathologies such as chronic inflammatory diseases or metabolic disorders including obesity or further diabetes of type 2. These imbalances are also potentially involved in the development of cancers and the setting into place of behavioral syndromes. Maintaining the balance of the intestinal microbiota is therefore essential: influencing its composition and/or its activity would be a major asset for treating the aforementioned syndromes. In this spirit, the idea of using so-called <<probiotic>> bacterial strains for intervening on pathologies influenced by the microbiota makes all its sense.

Defined in 2001 by the World Health Organization (WHO) and the United Nations Organization for Food and Agriculture (FAO), probiotics are <<living microorganisms, which, when they are ingested in a sufficient amount, exert positive effects on health, beyond traditional nutritional effects>>.

The present invention, as for it, is included in the re-establishment of conditions giving the possibility of optimizing juvenile growth and of restoring normal juvenile growth in a context of malnutrition.

The present invention thus has the goal of proposing new compositions giving the possibility of promoting juvenile growth or re-establishing normal juvenile growth in human or animal subjects, having been or being subject to malnutrition, notably undernourishment and/or poor assimilation.

Another goal of the invention is to provide such compositions, based on the use of bacterial strains having intestinal tropism, notably commensal strains, or acceptable as a probiotic.

Another goal of the invention is to provide such compositions which may also be included in a therapeutic background.

Still another goal of the invention is to provide a probiotic or therapeutic treatment method.

Still another goal of the invention is to provide such compositions and methods giving the possibility of making a health and/or nutritional claim in accordance with the legislation in effect, notably European legislation.

The invention is based on the fact that certain bacterial strains having intestinal tropism in an animal species have an effect promoting or re-establishing juvenile growth in a subject of this same species or of another species which is subject to malnutrition, notably to under-nourishment or poor assimilation. It was thus able to be demonstrated that bacterial strains of the Lactobacillus genus were capable of promoting juvenile growth both in a Drosophila (fruit fly) model, and in a mouse model coupled with poor nutritional diets and further a connection was able to be established between these results in mice and an increase in the serum level of IGF-1 in mice treated under these conditions with bacteria.

The object of the present invention is therefore a composition comprising at least one bacterial strain having intestinal tropism, in particular a lactic bacterium, for a use in order to promote juvenile growth in the case of malnutrition. The notion of malnutrition groups together under-nourishment, over-nourishment and poor assimilation. The invention is in particular directed towards under-nourishment and/or poor assimilation. It is preferably directed to under-nourishment. The indication of the composition may be a therapeutic or a health indication, as a drug, or nutritional indication, like a probiotic.

By bacterium having <<intestinal tropism>>, is meant a bacterium with the capability of passing through the gastric barrier and which is capable of persisting in the intestine.

According to an advantageous feature of the invention, the bacteria may promote the production of IGF-1 in humans or animals which are treated with the composition according to the invention.

The invention notably proposes bacterial strains belonging to the following families: Lactobacillaceae, Streptoccaceae, Enterococcaceae, Leuconostocaceae, Bifidobacteriaceae. According to one method, the invention uses one or more strains of the Lactobacillus genus, in particular one of the following species, Lactobacillus delbrueckii, Lactobacillus plantarum, Lactobacillus fermentum, Lactobacillus casei, Lactobacillus paracasei, Lactobacillus rhamnosus.

More particularly, these are bacteria belonging to the species Lactobacillus plantarum, Lactobacillus fermentum, Lactobacillus casei, Lactobacillus paracasei, Lactobacillus rhamnosus. According to one method, the strain is selected from the species Lactobacillus plantarum, Lactobacillus fermentum, Lactobacillus casei,

According to an embodiment, the bacterial strain is selected from among L. plantarum WJL, L. plantarum G821, L. casei ATCC 393, L. casei L919, L. paracasei ATCC25302, L. paracasei Shirota, L. fermentum ATCC9338, L. rhamnosus L900, L. rhamnosus L908, L. rhamnosus GG. According to one method, the strain is selected from L. plantarum WJL, L. plantarum G821, L. casei ATCC 393, L. casei L919, L. fermentum ATCC9338.

In a specific embodiment, these are bacteria of the species Lactobacillus plantarum, for example the WJL strain or the G821 strain, deposited at the <<Collection Nationale de Culture de Microorganismes (CNCM)>> (Institut Pasteur) under the registration number CNCM I-4979 on May 11, 2015. The G821 strain was obtained by experimental development (i.e. by accumulation and selection of natural variants) of the strain L. plantarum NIZO2877

According to a method, the compositions according to the invention comprise at least one bacterial strain selected from these groups, which has the required properties and gives the possibility of promoting or re-establishing juvenile growth in spite of the malnutrition period. Of course, the composition according to the invention may comprise more than one bacterial strain meeting the needs of the invention. Notably, the composition comprises two or more of these bacterial strains, selected from a same species or from different species.

According to an interesting embodiment, the bacterium is an L. plantarum. Suitable strains are L. plantarum G821 and L. plantarum WJL (Eun-Kyoung Kim et al., Genome Announcements, November/December 2013, Vol. 1, no. 6 e00937-13, GenBank AUTE00000000, Lactobacillus plantarum WJL, whole genome shotgun sequencing project). This WJL strain was initially isolated and may be isolated from the fruit fly (J H Ryu et al., Science 2008, 319: 777-782).

Other examples of suitable strains are the following: L. casei ATCC 393, L. casei L919 (Koryszewska-Baginska A. et al., Sep. 26, 2013, Genome Announc), L. paracasei ATCC25302, L. paracasei Shirota (Yuki N et al., Int J Food Microbiol. Apr. 1, 1999; 48 (1):51-7), L. fermentum ATCC9338, L. rhamnosus L900 (Aleksandrzak-Piekarczyk T. et al., Genome Announc, Aug. 15, 2013), L. rhamnosus L908 (Koryszewska-Baginska A. et al., Feb. 20, 2014, Genome Announc.), L. rhamnosus GG (Kankainen M. et al., Proc. Natl. Acad. Sci. USA, Oct. 6, 2009).

The present invention therefore contributes to the technique with the teaching that bacterial strains with intestinal tropism promote juvenile growth notably in poorly-fed (human or animal) patients. But the invention is not limited to this teaching, it further gives one skilled in the art the sure determination of the useful bacterial strains for the invention. Different criteria may be at the basis of tests, while being used alone or in association. From among these criteria, it will be noted that the serum IGF-1 level in the model animal (e.g. mouse), the larval growth in Drosophila melanogaster, the growth of model mice for example illustrated by the size of the femurs or the increase in size of the animals. On the basis of these criteria or of similar criteria, it is possible for one skilled in the art to develop tests comparing individuals raised in the presence or in the absence of the bacterium to be tested, under malnutrition conditions. The invention provides a significant addition by proposing axenic animal models and the raising of the animals in the presence or in the absence of the bacterium or bacteria to be tested giving the possibility of focussing the test on the analysis of the intrinsic properties of the tested strain(s) and thus doing without the effect that the residing intestinal microbiota of conventional animals may have within the context of the functional test. Increased predictivity of the test results therefrom as to the functional potential of the tested strain.

By <<axenic>> organism is meant an organism (e.g. a fruit fly, a mouse) raised in an environment without any microorganisms and therefore without any intestinal flora.

By <<monoxenic>> organism is meant an associated axenic organism (e.g. Drosophila, mouse) raised in the presence of a single microorganism and therefore bearing this single microorganism as intestinal flora.

According to a feature of the invention, the bacterial strains according to the invention are characterized by the fact they positively react to or were selected by using a test in which the growth of larvae from axenic Drosophila on a yeast-deficient nutritive medium (and therefore protein-deficient) is compared with that on a conventional nutritive medium. The laboratory conventional nutritional media generally comprise a minima a vegetable flour, typically maize flour, inactivated yeast, typically beer yeast or bread yeast (Saccharomyces cerevisiae), agar-agar and water. An inactivated yeast-deficient medium does not allow optimum post-embryo (i.e. juvenile) development of larvae of Drosophila, as this was reported in G. Storelli et al., Cell Metabolism 14, 403-414, 2011. A group of embryos is additionally sown with the bacterium to be tested, which gives the possibility to the larvae emerging from the embryos, of being associated with the bacterium to be tested. It is therefore possible to detect whether this bacterium may promote or allow juvenile growth in spite of a yeast-deficient nutritive medium.

The Drosophila test is typically conducted in the following way:
a batch of embryos issued from Drosophila melanogaster parents (e.g. the Drosophila yw strain, or any other axenic so-called <<wild>> strain);
on D1: these embryos are distributed into at least 2 groups (40 embryos, made in triplicate for a total of 120 embryos per group) on a nutritional medium comprising maize flour, agar-agar and water, deficient in yeast, one of the groups of embryos is further inoculated with a suspension of about 10⁸ CFUs of the bacterium to be tested in a saline buffer (e.g. PBS), which forms the monoxenic group, the other group forming the axenic group, the cultivation is conducted at about 25° C. until D7; typically the culture medium is made up, for 1 liter of medium, with 7.14 g of agar-agar, 80 g of maize flour and 6 g of inactivated yeast, the medium is cooked in boiling water for 10 mins, and then cooled; typically the same medium, non-deficient in inactivated yeast, contains 50 g of inactivated yeast;
on D7: at least 60 larvae of Drosophilas from each group either stemming from the inoculated embryos or not (monoxenic larvae and axenic larvae respectively) are recovered, a fast thermal shock is applied to them (e.g. 5 seconds; typically the larvae are placed for 5 seconds on a hob heated to 100° C.; this means killing the larvae without deforming them in order to allow measurement of their size);
the average length of the larvae of each of the groups is determined, and the obtained averages are compared;
the tested bacterial strain is considered as positively reacting to the test if the average of the length of the larvae of the monoxenic group is greater than the average of the length of the larvae of the axenic group with a value of p of less than or equal to 0.001, preferably less than or equal to 0.0001 in the Mann-Whitney statistical test, carried out on the whole of the set of data of the sizes of the larvae of both groups.

The object of the present invention is therefore a composition comprising at least one bacterial strain having intestinal tropism, in particular a lactic bacterium, for use in order to promote juvenile growth in the case of malnutrition, wherein the bacterial strain positively reacts to the test on a Drosophila as described above. As examples, mention may be made of the following strains: L. plantarum WJL, L. plantarum G821, L. casei ATCC 393, L. casei L919, L. paracasei ATCC25302, L. paracasei Shirota, L. fermentum ATCC9338, L. rhamnosus L900, L. rhamnosus L908, L. rhamnosus GG. Other strains may be identified from among bacteria with intestinal tropism and notably from among the species mentioned above.

In an embodiment of the invention, the WJL strain, or any other strain having a <<marked>> effect (for example L. casei ATCC 393, L. fermentum ATCC 9338, L. paracasei ATCC 25302) is used as a reference strain in order to identify and select the bacterial strains having a <<marked>> effect on the juvenile growth, i.e. an effect close to that of this reference strain, e.g. WJL (an effect not significantly different from the reference strain, e.g. WJL), or a <<strong>> effect on the juvenile growth (an effect significantly greater than that of the reference strain, e.g. WJL).

To do this, the Drosophila test (including 60 larvae per condition) is applied to the reference strain, e.g. WJL and to the strain to be tested (in parallel, preferably or else reference data may be available generated beforehand for the reference strain, e.g. WJL, for example the data shown in the examples). The averages obtained for both of the strains, is then compared. The tested bacterial strain is considered as being a strain with a marked effect if the average of the length of the larvae of the monoxenic group is not significantly different from the average of the length of the larvae of the reference group, e.g. WJL with a value of p greater than 0.001 in the Mann-Whitney statistical test, carried out on the whole of the set of data. The effect is strong if said average for the strain to be tested is significantly greater than the average for the reference strain, e.g. WJL, which is the case when the value of p of the statistical test is less than or equal to 0.001, preferably less than 0.0001. The effect is described as intermediate if said average for the strain to be tested is significantly less than the average for the reference strain, e.g. WJL, which is the case when the value of p of the statistical test is less than or equal to 0.001, preferably 0.0001.

The object of the present invention is therefore a composition comprising at least one bacterial strain having intestinal tropism, in particular a lactic bacterium, for the use of promoting juvenile growth in the case of malnutrition, wherein the bacterial strain has a marked or strong effect on the growth in the Drosophila model as described above. As an example, mention may be made of the following strains: L. plantarum WJL, L. plantarum G821, L. casei ATCC 393, L. casei L919, L. paracasei ATCC25302, L. paracasei Shirota, L. fermentum ATCC9338, L. rhamnosus L900, L. rhamnosus L908, L. rhamnosus GG. Other strains may be identified from among bacteria with intestinal tropism and notably from among the species mentioned above.

The object of the invention is notably a composition comprising a bacterial strain with intestinal tropism, notably belonging to the families, genera or species mentioned above, preferably to the Lactobacillus genus, for a use aiming at promoting juvenile growth in the case of malnutrition, wherein the bacterial strain positively reacts to the following test:
a batch of embryos from axenic Drosophila melanogaster parents is available;
on D1: these embryos are distributed into at least 2 groups (40 embryos, produced in triplicate i.e. 120 embryos per group) on a nutritional medium comprising maize flour, agar-agar and water, deficient in yeast, one of the groups of embryos is inoculated with a suspension of about 10⁸ CFUs of said bacterium in a saline buffer, which forms the monoxenic group test, the other group being inoculated with a suspension of about 10⁸ CFUs of the reference bacteria L. plantarum WJL, L. casei ATCC 393, L. fermentum ATCC 9338 or L. paracasei ATCC 25302, in a saline buffer, forming the monoxenic reference group, the cultivation of both groups is conducted at about 25° C. up to D7;
on D7: at least 60 larvae of Drosophila of each group are recovered, a thermal shock is applied to them;
the average of the length of the larvae of each of the groups is determined and the obtained averages are compared;
said bacterial strain having, as compared with the reference bacterium, a strong effect with an average of the length of the larvae of the monoxenic group test with this bacterial strain significantly greater than the average for the monoxenic group with the reference strain, with a value of p being less than or equal to 0.001, preferably 0.0001 in the Mann-Whitney statistical test, carried out on the whole of the set of data of the sizes of the larvae of both groups.

According to the invention, it is also possible to determine whether a bacterial strain with intestinal tropism gives the possibility of promoting growth in the case of malnutrition, by using an axenic mouse model which gives the possibility of performing a follow-up of skeletal growth of mice in the presence of the bacterium to be tested as compared with the absence of a microbiota and/or with the presence of a reference bacterial strain. This model may be used in first intention or on strains having positively reacted to the Drosophila test.

According to a feature of the invention, the bacterial strains according to the invention are characterized by the fact that they positively react to the following skeletal growth test:
from a same mouse line (typically Balb/c mice), a line of axenic parent mice and a line of monoxenic parent mice (associated with the bacterium to be tested) are established, and juveniles are produced which are raised with the parents in a conventional nutritional medium until their weaning (on D21); in order to form the group of monoxenic juveniles, mono-associated parents with the bacterial strain to be tested are used,
on D21: weaned juveniles stemming from each of both lines, forming the monoxenic group and the axenic group, are raised with a protein-deficient (8%) and lipid-deficient (2.5%) nutritional diet; typically the conventional nutritional diet comprises 23% of proteins and 5% of lipids,
on D56: the average of the sizes of the mice is determined for a relevant group by measuring the distance from the end of the snout to the base of the tail of each individual; another possible measurement consists of sacrificing the individuals, and of sampling the femurs and measuring the latter.
the lactic bacterium strain being considered as positively reacting to the test if the average size of the individuals and/or of the femurs, of the monoxenic group is greater than the average size, of the individuals and/or femurs respectively, of the axenic group, with a value of p of less than or equal to 0.05, in Tukey's statistical test.

The object of the present invention is therefore a composition comprising at least one bacterial strain having intestinal tropism, in particular a lactic bacterium, for use in order to promote juvenile growth in the case of malnutrition, wherein the bacterial strain positively reacts to the skeletal growth test on mice. The Drosophila test allows more rapid screening, so that generally, the test on Drosophilas is also available, and the bacterial strain positively reacts to Drosophila and skeletal growth tests. As an example, mention may be made of the following strain: L. plantarum WJL. Other strains may be identified from among bacteria with intestinal tropism and notably from among the species and strains mentioned above, notably among the strains L. plantarum G821, L. casei ATCC 393, L. casei L919, L. paracasei ATCC25302, L. paracasei Shirota, L. fermentum ATCC9338, L. rhamnosus L900. L. rhamnosus L908, L. rhamnosus GG.

In an embodiment of the invention, the strain WJL or another strain with a <<marked>> effect is used as a reference strain in order to identify and select bacterial strains having a <<marked>> effect on juvenile growth, i.e. an effect close to one of this reference strain, e.g. WJL (an effect not significantly different from the reference strain, e.g. WJL), or a <<strong>> effect on juvenile growth (an effect significantly greater than that of the reference strain, e.g. WJL).

To do this, the mouse test (including 8 mice per condition) is applied to the reference strain, e.g. WJL and to the strain to be tested (in parallel, preferably or else reference data may be available generated beforehand for the strain WJL, for example data shown in the examples). The averages obtained for both strains are then compared. The tested bacterial strain is considered as being a strain with a marked effect if the average of the average size of the individuals and/or of the femurs of the monoxenic group is not significantly different from the corresponding average for the reference group, e.g. WJL with a value of p greater than 0.05 in Tukey's statistical test. The effect is strong if said average for the strain to be tested is significantly greater than the average for the reference strain, e.g. WJL, which is the case when the value of p of the statistical test is less than or equal to 0.05. The effect is described as intermediate if said average for the strain to be tested (which was described with respect to the axenic mice in the preceding test) is significantly less than the average for the reference strain, e.g. WJL, which is the case when the value of p of the statistical test is less than or equal to 0.05.

The composition according to the invention will preferably comprise a bacterial strain having such a marked or strong effect.

The bacterial strains according to the invention may be characterized by the fact that they have a positive impact on the IGF-1 serum level. It was in this way that it was possible, on the basis of a model of axenic mice, to show that mice raised in a medium with a low protein content but in the presence of the bacterium (monoxenic mice) had greater growth and at the same time a greater IGF-1 serum level, as compared with these same mice raised with this protein-deficient medium and in the absence of the bacterium (axenic mice). This gives the possibility of proposing a test allowing determination whether a bacterial strain has the potential of increasing the IGF-1 serum level, this test may be applied in association with a skeletal growth test in order to specify or refine the effect of the strain in this context of juvenile growth, or in a first intention.

In this case, the bacterial strains according to the invention are characterized by the fact that they positively react to the following IGF-1 serum level test:
from a same line of mice (typically Balb/c mice), a line of axenic parent mice and a line of monoxenic parent mice (associated with the bacterium to be tested) are established, and juveniles are produced which are raised with the parents in a conventional nutritional medium until their weaning (on D21); in order to form the group of monoxenic juveniles, mono-associated parents with the bacterial strain to be tested are used,
on D21: 8 weaned juveniles are available issued from each of both of these lines, forming the monoxenic group and the axenic group, and they are raised with a protein-deficient (8%) and lipid-deficient (2.5%) nutritional diet; typically, the conventional nutritional diet comprises 23% of proteins and 5% of lipids,
on D56: blood is taken from the juveniles of each group and the average IGF-1 serum level is determined for each group; this measurement of the IGF-1 serum level is preferably conducted on diluted serum (1:25); commercial ELISA kits for detecting IGF-1 are used by following the instructions of the manufacturer,
the lactic bacterial strain is considered as positively reacting to the test if the average IGF-1 serum level of the monoxenic group is greater than the average serum level of the axenic group with a value of p of less than or equal to 0.05 in the Tukey statistical test.

The object of the present invention is therefore a composition comprising at least one bacterial strain having intestinal tropism, in particular a lactic bacterium, for use in order to promote juvenile growth in the case of malnutrition, wherein the bacterial strain increases the IGF-1 serum level. This is notably a bacterial strain which positively reacts to the test on mice as described above.

The object of the present invention is also a composition comprising at least one bacterial strain having intestinal tropism, in particular a lactic bacterium, for use in order to promote juvenile growth in the case of malnutrition, wherein the bacterial strain positively reacts to the skeletal growth test on mice and increases the IGF-1 serum level in these same mice, notably said strain positively also reacts to the test on Drosophila.

As an example, mention may be made of the following strain: L. plantarum WJL. Other strains may be identified from among bacteria with intestinal tropism and notably from among the species and strains mentioned above, notably from among the strains L. plantarum G821, L. casei ATCC 393, L. casei L919, L. paracasei ATCC25302, L. paracasei Shirota, L. fermentum ATCC9338, L. rhamnosus L900. L. rhamnosus L908, L. rhamnosus GG.

In an embodiment of the invention, the strain WJL or another strain with a <<marked>> effect is used as a reference strain in order to identify and select bacterial strains having a <<marked>> effect on the IGF-1 serum level, i.e. an effect close to the one of this reference strain, e.g. WJL (an effect not significantly different from the reference strain, e.g. WJL), or a <<strong>> effect on the IGF-1 serum level (an effect significantly greater than the reference strain, e.g. WJL).

To do this, the mice test (including 8 mice per condition) for measuring the IGF-1 serum level is applied to the reference strain, e.g. WJL and to the strain to be tested (in parallel, preferably, or else reference data may be available generated beforehand for the reference strain, e.g. WJL, for example the data shown in the examples). The averages of the IGF-1 serum levels obtained for both strains are then compared. The tested bacterial strain is considered as being a strain with a marked effect if the average of the IGF-1 levels of the monoxenic group is not significantly different from the average for the reference group, e.g. WJL with a value of p greater than 0.05, in Tukey's statistical test. The effect is strong if said average for the strain to be tested is significantly greater than the average for the reference strain, e.g. WJL, when the value of p is less than or equal to 0.05. The effect is described as intermediate if said average for the strain to be tested (described beforehand in the preceding axenic test) is significantly less than the average for the reference strain, e.g. WJL, when the value of p is less than or equal to 0.05. The composition according to the invention will therefore preferably comprise a bacterial strain having such a marked or strong effect, and in particular this bacterial strain has a marked or strong effect both on skeletal growth and on the IGF-1 serum level.

The composition according to the invention, comprising at least one bacterial strain according to the invention, is able to be used as a drug or probiotic intended to promote juvenile growth in the case of malnutrition, notably with protein-deficiency. According to a feature of the invention, the subject to be treated, poorly fed or having been poorly fed, may have an IGF-1 serum level less than the one encountered in individuals of the same age and gender and not poorly fed.

The composition may be used in humans from birth to puberty.

The composition may be used in mammals, notably productive livestock (cattle, sheep, goats, pigs, poultry), pet animals (dog, cat) and sport animals (horse, dromedary, camel), between weaning and sexual maturity.

The composition contains an amount from 10⁵ to 10¹², notably from 10⁶ to 10¹², preferably from 10⁸ to 10¹² bacterial cells forming colonies (CFU) according to the invention, per gram of composition. The term CFU means colony forming units>> according to the English expression. By gram of composition, is preferably meant the pharmaceutical composition or the probiotic composition formed with bacteria, co-ingredients, and excipients or carriers. By bacterial cells, is meant a unique bacterial strain according to the invention or a mixture of at least two bacteria, according to the invention.

The composition comprises lactic bacteria(um) in a living form. This may be a bacterial suspension, which may be frozen and thawed after use, or further a freeze-dried power, which may be used as such or after resumption in a suitable carrier. It may then comprise a conventional freeze-drying excipient.

The composition may be an oral administration dosage form (for example a powder, a gelatin capsule, a tablet) optionally in a gastro-protected dosage form giving the possibility of crossing the stomach and of releasing the bacteria in the intestine.

Also the object of the invention is a method for probiotic treatment or therapeutic treatment for promoting juvenile growth in the case of malnutrition, comprising administration to a human (from birth to puberty) or animal (from weaning to sexual maturity) patient in need thereof, i.e. poorly fed or having been subject to a malnutrition period, notably with protein-deficiency, of a composition according to the invention. The patient in need thereof may have an IGF-1 serum level less than the one encountered in individuals of the same age and gender and not poorly fed. As indicated, the method may be a therapeutic treatment or a probiotic treatment (or a nutritional supplement). The method comprises administration of a sufficient amount of a composition as described above. The amount and the frequency of administration will notably depend on the severity of the malnutrition, on the age and on the condition of the patient. The method will comprise administration in one or several times, which may be staged over the growth period of the subject (up to puberty or sexual maturity), of doses of the composition according to the invention. The doses may be fractionated for facilitating administration, notably depending on the age of the patient. The administration frequency is notably comprised between one dose (single or fractionated) every day and one dose every month. Typically, the administration frequency will be comprised between one dose (single or fractionated) daily and one dose monthly, or even every 2, 3, 4, 5 or 6 days. Each dose (single or fractionated) notably represents several grams to several tens of grams of composition. The method notably comprises administration of a composition comprising an amount from 10⁵ to 10¹², notably from 10⁶ to 10¹², preferably from 10⁸ to 10¹² bacterial cells forming colonies (CFU) per gram of composition (by bacterial cells, is meant a unique bacterial strain according to the invention or a mixture of at least two bacteria according to the invention). The method notably comprises the administration of a composition comprising the lactic bacteria(um) in a live form. This composition may be a bacterial suspension, which may be frozen and thawed out before use, or further a freeze-dried powder, which may be used as such or after resumption in a suitable carrier. This composition may then comprise a conventional freeze-drying excipient, be an oral administration dosage form (for example, a powder, a gelatin capsule, a tablet) optionally in a gastro-protected form giving the possibility of crossing the stomach and releasing the bacteria in the intestine, or further a rectal form (for example a suppository).

In an embodiment, the composition according to the invention, notably the composition used in the treatment method, comprises at least one bacterial strain which is not naturally occurring in the species to which belongs the patient to be treated. Thus, this may be a bacterium which is not naturally present in humans or in animals. In this configuration, when there are several different bacteria, it is sufficient that one of them is not naturally present. On the other hand, this bacterium proves to have intestinal tropism in the patient to be treated or in the corresponding species (a human or animal according to the invention) and fits the definition of active strains according to the invention.

The invention also relates to a method for screening bacteria capable of promoting juvenile growth in the case of malnutrition, using a model of an axenic fruit fly and/or a model of an axenic mouse.

The method with the Drosophila model comprises the following steps:
a batch of embryos stemming from Drosophila melanogaster (e.g. the strain Drosophila yw or any other so-called <<wild>> strain) which are axenic is available;
these embryos are distributed into at least 2 groups on a nutritional medium comprising a nutritive medium for Drosophila which medium is yeast-deficient, one of the groups of embryos is further inoculated with a suspension of the bacterium to be tested, which forms the group of monoxenic individuals; the other group forms the axenic group,
the cultivation is conducted at a suitable temperature (generally about 25° C.),
at the end of a suitable cultivation period (generally about 7 days), the larvae of Drosophila of each group stemming from the either initially inoculated embryos or not (axenic larvae and monoxenic larvae) are recovered, preferably recovered in an equal number,
the average of the length of the larvae of each of the groups, is determined and the obtained averages are compared;
the tested bacterial strain is considered as positively reacting to the test if the average of the length of the larvae of the monoxenic group is greater than the average of the length of the larvae of the axenic group with a value of p of less than or equal to 0.001, preferably less than or equal to 0.0001 in the Mann-Whitney statistical test, performed on the whole of the set of data of the sizes of the larvae of both groups.

Preferably, the screening method takes over the features of the Drosophila test which was described above.

The method with the mice model comprises the following steps:
juveniles issued from two lines issued from a same mouse strain (typically Balb/c mice), are available, i.e. a line of axenic parent mice and a line of monoxenic parent mice (associated with the bacterium to be tested),
they are cultivated with a protein-deficient and preferably also lipid-deficient nutritional diet,
at the end of a suitable raising period, the average of the values of one or several parameters of each group is determined, these parameters being related to growth (for example increase in weight, skeletal growth for example by measuring the size of the individuals or the size of their femurs), and/or to the IGF-1 serum level,
the lactic bacterial strain is considered as positively reacting to the test if the average value of the measured parameter of the monoxenic group is greater than the average value of the measured parameter of the axenic group with a value of p of less than or equal to 0.05 in Tukey's statistical test.

Preferably, the screening method takes up the features of the mice skeletal growth test or of the IGF-1 serum level test described above.

The screening method may also be a comparative test with a reference strain, for example the strain WJL, and this test then takes up the features described above for the Drosophila or mice tests.

The object of the present invention is also a pure culture or an isolate of the G821 strain (CNCM I-4979), in a live or killed form, a composition comprising this living strain in a preservation or culture medium, a freeze-dried composition comprising this strain and optionally a freeze-drying excipient and/or a preservative, a composition comprising this strain in a killed form in a preservation medium, or killed in a preservation medium, a composition which may be administered to humans or animals, comprising this living or killed strain in a medium or excipient acceptable for oral or rectal administration. The preservation, culture media, the freeze-drying excipients, the preservatives, the media or excipients acceptable for oral or rectal administration are those described above and those well known to one skilled in the art.

The invention will now be described in more details by means of the embodiments of the invention taken as non-limiting examples and referring to the drawing wherein:

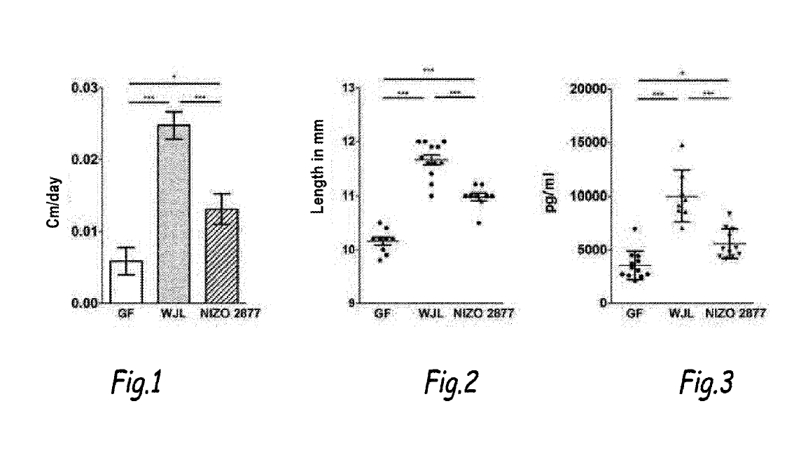

FIG. 1 is a graph showing the growth rate (in cm/day) of mice from D28 to D56; GF: axenic mice; WJL: mice bred in the presence of the strain L. plantarum WJL; NIZO 2877: bred mice in the presence of the strain L. plantarum WJL NIZO 2877; * p<0.05; ***p<0.001;

FIG. 2 is a graph showing the size of femurs on D56, in GF mice, WJL or NIZO 2877; * p<0.05; ***p<0.001;

FIG. 3 is a graph showing the serum levels (in ng/ml) of male mice on D56, groups GF, WJL or NIZO 2877* p<0.05; ***p<0.001;

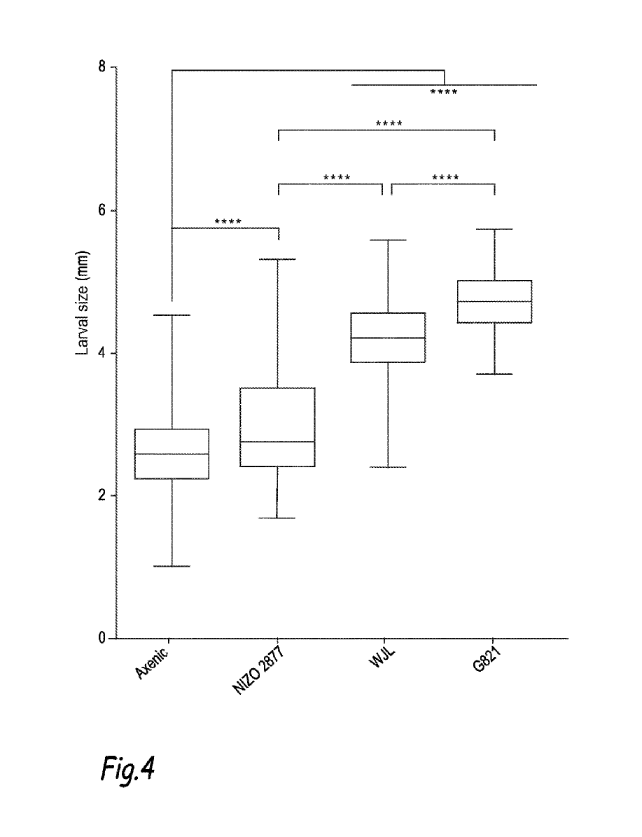

FIG. 4 is a graph showing the size of larvae of Drosophila at 7 days under conditions of undernourishment: a negative control group (no addition of bacteria on the growth medium after laying), and groups characterized by the addition on the growth medium of the strain L. plantarum NIZO 2877, WJL or G821.

### EXAMPLE 1

### Functional Screening of Bacteria on Monoxenic Drosophila

D-1: adult Drosophilae are used (axenic D. melanogaster yw); they are placed in laying cages including a bottom (of the Petri dish type) on which is placed a conventional nutritional medium; laying is ensured by maintaining the adult population in the cage for 1 night at 25° C.

D0: 6 parts of the nutritional medium are cut out so as to collect 6×40 embryos, each of the parts are placed on a deficient nutritional medium contained in a Petri dish, three boxes are inoculated with sterile PBS 1× and then the three other ones with a suspension of the bacterium in PBS 1×.

Incubation is performed for 7 days at 25° C.

D7: Next, from each box, the larvae which have developed therein (the experiment may be utilized from 20 larvae per box, we therefore used hereafter groups of more than 60 individuals) are recovered. The larvae undergo a shock thermal treatment (5 seconds on a hob heated to 100° C.), the size of the larvae is then measured.

This procedure was applied to different bacterial strains, and the experiments and results are summarized in the following Table 1:

TABLE 1

| | Sizes of the larvae | | | | | | |
|---|---|---|---|---|---|---|---|
| | at D7 (mm) | | Statistical analysis | | | | |
| | Average | | Comparison/axenic animals | | Comparison/L. plantarum WJL | | Interpretation of the |
| | (n > 60. | Standard | Value of p | Statistical | Value of p | Statistical | qualitative test |
| | mm) | deviation | (1) | significance | (1) | significance | (quantitative level) |
| Axenic | 2.693 | 0.497 | | | <0.0001 | **** | |
| Lactobacillus plantarum WJL | 3.644 | 0.883 | <0.0001 | **** | | | GPS (marked) |
| Lactobacillus plantarum NIZO2877 | 3.398 | 0.750 | <0.0001 | **** | 0.0001 | *** | GPS (intermediate) |
| Lactobacillus casei ATCC 393 | 4.391 | 0.802 | <0.0001 | **** | 0.0721 | ns | GPS (marked) |
| Lactobacillus fermentum ATCC 9338 | 4.133 | 0.824 | <0.0001 | **** | >0.9999 | ns | GPS (marked) |
| Lactobacillus fermentum KLD | 2.741 | 0.626 | >0.9999 | ns | <0.0001 | **** | No effect on growth |
| Lactobacillus fermentum LMG | 3.287 | 0.799 | 0.1423 | ns | 0.0015 | ** | No effect on growth |
| Lactobacillus paracasei ATCC 25302 | 3.987 | 0.524 | <0.0001 | **** | 0.4258 | ns | GPS (marked) |
| Lactobacillus paracasei BL23 | 2.682 | 0.482 | >0.9999 | ns | <0.0001 | **** | No effect on growth |
| Lactobacillus paracasei Shirota | 3.407 | 0.779 | <0.0001 | **** | 0.0984 | ns | GPS (marked) |
| Lactobacillus delbrueckii | 2.540 | 0.599 | >0.9999 | ns | <0.0001 | **** | No effect on growth |
| spp. bulgaricus ATCC 11842 | | | | | | | |
| Lactobacillus casei L919 | 4.150 | 0.712 | <0.0001 | **** | 0.2966 | ns | GPS (marked) |
| Lactobacillus rhamnosus L900 | 3.920 | 0.572 | <0.0001 | **** | >0.9999 | ns | GPS (marked) |
| Lactobacillus rhamnosus L908 | 4.112 | 0.576 | <0.0001 | **** | 0.1227 | ns | GPS (marked) |
| Lactobacillus rhamnosus GG | 4.036 | 0.488 | <0.0001 | **** | 0.5272 | ns | GPS (marked) |

*Significantly greater than the reference value
*Significantly less than the reference value
GPS = Growth promoter strain

(1) Statistical Test Used: Mann-Whitney Test.

### Indications on the Validation Criteria of the <<Larval Growth>> Phenotype Test

The strain should at least fulfill criterion 1 in order to be validated and the extent of its activity may be described according to the criterion 1+2.

Qualitative Criterion 1=Growth Promoting Strain Relatively to the Axenic Condition: A statistical test is applied to the whole of the values of length of the individuals associated relatively to the values of the axenic individuals (at least 60 larvae per condition). A value p of less than 0.001 (Mann-Whitney test) describes the strain as “growth promoting”. In the present case, the following strains were described as growth-promoting:

Lactobacillus plantarum WJL
Lactobacillus plantarum NIZO2877
Lactobacillus casei ATCC 393
Lactobacillus fermentum ATCC 9338
Lactobacillus paracasei ATCC 25302
Lactobacillus paracasei Shirota
Lactobacillus casei L919
Lactobacillus rhamnosus L900
Lactobacillus rhamnosus L908
Lactobacillus rhamnosus GG

The following strains do not have any growth-promoting effect:

Lactobacillus fermentum KLD
Lactobacillus fermentum LMG
Lactobacillus paracasei BL23
Lactobacillus delbrueckii spp. bulgaricus ATCC 11842

Quantitative Criterion 2=Extent of the Growth Promoting Effect (with Criterion 1 Having been Validated Beforehand):

A statistical test is applied to the whole of the values of length of the individuals associated with the tested strain(s) relatively to the values of the mono-associated individuals with the strain L.plantarum WJL (at least 60 larvae per condition).

Three scenarii occur: The value p of the statistical test is greater than 0.001 (Mann-Whitney test), the strain will be described as having a <<marked>> effect on the larval growth (similar effect to the strain L.plantarum WJL).

In the present case, the following strains have a marked effect:

Lactobacillus casei ATCC 393
Lactobacillus fermentum ATCC 9338
Lactobacillus paracasei ATCC 25302
Lactobacillus paracasei Shirota
Lactobacillus casei L919
Lactobacillus rhamnosus L900
Lactobacillus rhamnosus L908
Lactobacillus rhamnosus GG
Lactobacillus plantarum WJL

The value p of the statistical test is less than 0.001 (Mann-Whitney test), the strain will be described as having an “intermediate” effect on larval growth if the average size of the individuals is less than those of the individuals associated with L.plantarum WJL (case of the strain L.plantarum NIZO 2877 in the presented example). The strain will be described as having a “strong” effect if the average size of the individuals is greater than those of the individuals associated with L.plantarum WJL (a non-identified case in the present example).

Analysis of the <<Larval Growth>> Phenotype Test Example:
14 Lactobacillus strains were tested (Table 1) in order to determine their growth promoting potential by means of the <<larval growth>> test in Drosophila melanogaster.
These strains are available from ATCC, at the Institut Pasteur of Paris, at the Institut Pasteur of Lille or published in the scientific literature and available from the referee researchers of the mentioned publications (Table 2).

TABLE 2

| | Public collection and/or publication |
|---|---|
| | with sequence of the genome |
| Lactobacillus plantarum | Kim et al., Genome Announc. Nov. 21, 2013, |
| WJL | 1(6). Pil: e00937-13 |
| Lactobacillus plantarum | NIZO (2877) |
| NIZO2877 | |
| Lactobacillus casei | ATCC (393) |
| ATCC 393 | |
| Lactobacillus fermentum | ATCC (9338) |
| ATCC 9338 | |
| Lactobacillus fermentum | Institut Pasteur of Lille (A5.20) |
| KLD | |
| Lactobacillus fermentum | Institut Pasteur of Lille (A5.16) |
| LMG | |
| Lactobacillus paracasei | ATCC (25302) |
| ATCC 25302 | |
| Lactobacillus paracasei | Institut Pasteur of Lille (A3.6) et Mazé et al., |
| BL23 | J. Bacteriol., May 2010; 192(10): 2647-8 |
| Lactobacillus paracasei | Institut Pasteur of Lille (A3.5) |
| Shirota | |
| Lactobacillus delbrueckii | ATCC (11842) et van de Guchte M, et al., |
| spp. bulgaricus | Proc. Natl Acad Sci USA Jun. 13, 2006 |
| Lactobacillus casei | Koryszewska-Baginska A et al., Genome |
| L919 | Announc, Sep. 26, 2013 |
| Lactobacillus rhamnosus | Aleksandrzak-Piekarczyk T et al. Genome |
| L900 | Announc, Aug. 15, 2013 |
| Lactobacillus rhamnosus | Koryszewska-Baginska A et al. Genome |
| L908 | Announc, Feb. 20, 2014 |
| Lactobacillus rhamnosus | ATCC (53103) and Kankainen M et al., |
| GG | Proc Natl Acad Sci USA, Oct. 6, 2009 |

The size of a minimum of 60 individuals per condition is studied on day 7 after association with the bacterial strain to be tested, and then cultivation of the juvenile individuals in a yeast-deficient nutritional medium. Table 1 shows the average and the standard deviation of these sets of data.
The statistical analysis of these results gives the possibility of qualifying according to criterion 1 of the <<larval growth test>>, 10 strains of different species of Lactobacilli. This selection brings to light a strictly strain-specific functional effect and is not necessarily related to a given species of Lactobacillus.
Statistical analysis of the results gives the possibility according to criterion 2, of describing as <<intermediate>> the effect of the strain L.plantarum NIZO 2877 while the 9 other strains promoting growth have a <<marked>> effect.

### Example 2

### Description of a Strain with a Strong Effect on the Drosophila Model

The Drosophila test of Example 1 is reproduced with 3 bacterial strains:
Lactobacillus plantarum WJL
Lactobacillus plantarum NIZO2877
Lactobacillus plantarum G821

The analysis of the results (see FIG. 4) shows the following effects:

Axenic versus NIZO 2877 p<0.001 (***)
NIZO 2877 versus WJL p<0.0001 (****)
G821 versus WJL p<0.0001 (****)
Axenic versus WJL ou G821 p<0.0001 (****)

### Conclusion:

Lactobacillus plantarum WJL: marked effect
Lactobacillus plantarum NIZO2877: intermediate effect
Lactobacillus plantarum G821: strong effect.

### Example 3

### Functional Screening of Bacteria on Mice—Phenotype Analysis

Study in mice of the juvenile growth promoting effect of two strains with <<intermediate>> and <<marked>> effect (L. plantarum NIZO2877 and L.plantarum WJL, respectively) identified by means of the <<larval growth>> test of Example 1.

The male progeny (a minimum of 15 individuals) of three groups of individuals stemming from a same axenic mouse colony was studied, the first group consists in axenic juveniles (group GF), the second one in juveniles issued from mono-associated parents with the strain L.plantarum WJL (group WJL), and the third one in juveniles stemming from mono-associated parents with the strain L.plantarum NIZO2877 (group NIZO 2877). The parents and juveniles are cultivated in a conventional nutritional medium until weaning of the juveniles (J21), and then the weaned juveniles are cultivated in a protein-deficient and lipid-deficient (8% and 2.5% respectively) nutritional medium up to D56.
Conventional nutritional medium: 23% proteins and 5% lipids.
Deficient nutritional medium: 8% proteins and 2.5% of lipids.

Three parameters illustrating the juvenile growth of these individuals were studied: (1) the increase in size for a period of 28 days following weaning (from D28 to D56), (2) the size of the femurs of a batch of individuals (a minimum of 9 individuals) representative of the tested population at D56 and finally (3) the serum level at D56 of the IGF-1 growth factor in at least 8 individuals.

The statistical analyses were carried out by using Tukey's test by using the piece of software GraphPad (GraphPad Prism 5.04, San Diego, USA); values of p<0.05 are considered as significant.

(1) Increase in Size:

The mice were anesthetized by brief exposure to isoflurane in order to allow measurement of the size of the mice (from the snout to the base of the tail) on D28 and D56.

TABLE 3
growth rate (size increase per day in cm from D28 to D56) -
Nb = number of mice

| GF | | | WJL | | | NIZO 2877 | | |
|---|---|---|---|---|---|---|---|---|
| | Standard | | | Standard | | | Standard | |
| Average | deviation | Nb | Average | deviation | Nb | Average | deviation | Nb |
| 0.007321 | 0.009878 | 15 | 0.031114 | 0.009376 | 17 | 0.016429 | 0.011071 | 25 |

These data are collected in FIG. 1.

(2) Size of the Femurs:

The mice were sacrificed on D56, a femur was sampled, released from the muscle and its length was measured with the Vernier caliper.

TABLE 4
Length of the femurs in mm on D56 in males

| GF | WJL | NIZO 2877 |
|---|---|---|
| 10.2 | 11.2 | 10.9 |
| 10.2 | 11.6 | 11 |
| 10.5 | 11.9 | 10.5 |
| 9.9 | 11.6 | 11.2 |
| 10.4 | 12 | 11 |
| 10 | 11.4 | 11 |
| 10.2 | 12 | 11 |
| 10.2 | 11.7 | 11.2 |
| 9.8 | 12 | 11 |
| | 11.9 | |
| | 11.6 | |
| | 11 | |

These data are collected in FIG. 2.

(3) Serum Titers of IGF-1:

The IGF-1 titers are measured on the serum obtained from the blood of the sacrificed mice at D56. The measurement is conducted on diluted serum (1:25) by using the ELISA Ready-Set-Go kit (eBioscience, USA), by following the instructions of the manufacturer.

TABLE 5
Serum IGF-1 titer in pg/ml (dual measurement from ELISA plates)

| GF | | WJL | | NIZO 2877 | |
|---|---|---|---|---|---|
| 4526.75 | 4343.75 | 8823.25 | 8606.5 | 7291.25 | 6998.75 |
| 2825.5 | 2614.75 | 9258.5 | 9122.25 | 5128 | 5102 |
| 2667.5 | 2773 | 8471.25 | 8660.5 | 4343.75 | 4396 |
| 3455.25 | 3219.5 | 7025.25 | 7078.25 | 5232.75 | 5180.5 |
| 7131.5 | 6839.5 | 11063 | 9557.3 | 8471.25 | 8282.5 |
| 2164.2 | 2057.55 | 15125 | 14409 | 4866.5 | 4761.75 |
| 3140.75 | 3035.75 | 10023 | 9372 | 6363.25 | 6152.25 |
| 2509 | 2456.15 | 11735 | 12026 | 7211.25 | 6680.5 |
| 2588.25 | 2614.75 | | | 4213.25 | 4161 |
| 4432.5 | 4601.3 | | | 4770.5 | 4517 |
| 4013.3 | 3930 | | | 4940.3 | 3846.5 |
| 3597.3 | 3846.5 | | | | |

These data are collected in FIG. 3.

The results illustrate a <<marked>> effect of the strain L.plantarum WJL on the growth rate (value p<0.001 relative to the axenic condition) and an <<intermediate>> effect of the strain L.plantarum NIZO2877 (value p<0.05 relatively to the axenic condition and value p<0.001 relatively to the condition L.plantarum WJL). These effects are confirmed with the <<IGF-1 level>> parameter (same ranges of p values of the different tests), but reinforced with the <<femur size>> parameter (value p<0.001 between the axenic conditions and L.plantarum NIZO 2877).

The whole of these results demonstrates by scientific proof, the juvenile growth promoting effect of certain strains of Lactobacilles also positively reacting to the <<larval growth>> test.

All the public documents quoted here are incorporated by reference. Also, one skilled in the art may refer to these diverse documents and to the deposits of commercial strains to which reference is made here.

1. The composition comprising at least one Lactobacillus strain with intestinal tropism, selected from among the species Lactobacillus plantarum, Lactobacillus fermentum, Lactobacillus casei, for a use aiming at promoting juvenile growth in the case of malnutrition.

2. The composition according to claim 1, wherein the Lactobacillus strain is selected from among L. plantarum WJL, L. plantarum G821, L. casei ATCC 393, L. casei L919, L. fermentum ATCC9338.

3. The composition according to claim 14, wherein the Lactobacillus strain positively reacts to the following test:
a batch of embryos from axenic Drosophila melanogaster parents is available;
on D1: these embryos are distributed into at least 2 groups (40 embryos, produced in triplicate i.e. 120 embryos per group) on a nutritional medium comprising maize flour, agar-agar and water, deficient in yeast, one of the embryo groups is further inoculated with a suspension of about 10⁸ CFUs of the bacterium to be tested in a saline buffer, which forms the monoxenic group, the other group being the axenic group, the breeding of both groups is conducted at about 25° C. up to D7;
on D7: at least 60 Drosophila larvae of each group are recovered, a thermal shock is applied to them;
the average of the length of the larvae of each of the group is determined and the obtained averages are compared;
the tested bacterial strain is considered as positively reacting to the test if the average of the length of the larvae of the monoxenic group is greater than the average of the length of the larvae of the axenic group with a value p less than or equal to 0.001 in the Mann-Whitney statistical test, conducted on the whole of the set of data of the sizes of the larvae of both groups.

4. The composition according to claim 3, comprising a bacterial strain which, relatively to the strain L. plantarum WJL, in this same test on Drosophila, has a strong effect with an average of the length of the larvae of the monoxenic group with this bacterial strain significantly greater than the average for the monoxenic group with the strain WJL, when the value of p is less than or equal to 0.001 in the Mann-Whitney statistical test, conducted on the whole of the set of data of the sizes of the larvae of both groups.

5. The composition according to claim 1, wherein the Lactobacillus strain positively reacts to the following test:
from a same line of mice, a line of axenic parent mice is established and a line of monoxenic parent mice (associated with the bacterium to be tested), and juveniles are produced which are bred with the parents in a conventional nutritional medium up to their weaning (D21);
on D21: 8 weaned juveniles issued from each of these two lines are available, forming the monoxenic group and the axenic group, and they are raised in a protein-deficient (8%) and lipides (2.5%) nutritional diet;
on D56: the average of the sizes of the mice for each relevant group is determined by measuring the end of the snout to the base of the tail of each individual, and the femurs of the individuals of each group are taken and the latter are measured;
the lactic bacterium strain being considered as positively reacting to the test if the average size of the individuals or of the femurs of the monoxenic group is greater than the average size of the individuals or of the femurs, of the axenic group, with a value of p of less than or equal to 0.05 in the Tukey statistical test.

6. The composition according to claim 5, comprising a bacterial strain which, relatively to the strain L. plantarum WJL, in this same test on mice, has a strong effect with an average of the size of the mice or of their sizes of the monoxenic group with this bacterial strain significantly greater than the average for the monoxenic group with the strain WJL, when the value of p is less than or equal to 0.05 in the Tukey statistical test.

7. The composition according to claim 1, wherein the Lactobacillus strain positively reacts to the following test:
from a same line of axenic mice, a line of axenic parent mice and a line of monoxenic parent mice (associated with the bacterium to be tested) are established, and juveniles are produced which are raised with the parents in a conventional nutritional medium up to their weaning (D21),
on D21: 8 weaned juveniles are available stemming from each of both of these lines, forming the monoxenic group and the axenic group, and they are raised in a protein-deficient (8%) and lipid-deficient (2.5%) nutritional diet;
on D56: blood of the juveniles of each group is sampled and the average IGF-1 serum level is determined for each group;
the lactic bacterial strain being considered as positively reacting to the test of the average IGF-1 serum level of the monoxenic group is greater than the average serum level of the axenic group with a value of p of less than or equal to 0.05 in the Tukey's statistical test.

8. The composition according to claim 7, comprising a bacterial strain which, relatively to the strain L. plantarum WJL, in this same test on mice, has a strong effect with an average IGF-1 serum level of the monoxenic group with this bacterial strain significantly greater than the average for the monoxenic group with the strain WJL, when the value of p is less than or equal to 0.05 in the Tukey statistical test.

9. A composition comprising a bacterial strain with intestinal tropism for a use aiming at promoting juvenile growth in the case of malnutrition, wherein the bacterial strain positively reacts to the following test:
a batch of embryos of axenic Drosophila melanogaster parents;
on D1: these embryos are distributed into at least 2 groups (40 embryos, produced in triplicate i.e. 120 embryos per group) on a nutritional medium comprising maize flour, agar-agar and water, deficient in yeast, one of the groups of embryos is inoculated with a suspension of about 10⁸ CFUs of said bacterium in a saline buffer, which forms the monoxenic group test, the other group being inoculated with a suspension of about 10⁸ CFUs of a reference bacterium L. plantarum WJL, L. casei ATCC 393, L. fermentum ATCC 9338 or L. paracasei ATCC 25302, in a saline buffer, forming the monoxenic reference group, the raising of both groups is conducted at about 25° C. until D7;
on D7: at least 60 Drosophila larvae of each group are recovered, a thermal shock is applied to them;
the average of the length of the larvae of each of the groups is determined, and the obtained averages are compared;
said bacterial strain having, relatively to the reference bacterium, a strong effect with an average of the length of the larvae of the monoxenic group test with this bacterial strain significantly greater than the average for the monoxenic group with the reference strain, with a value of p of less than or equal to 0.001, in the Mann-Whitney statistical test, conducted on the whole of the set of data of the sets of larvae of both groups.

10. The composition according to claim 1, for use as a drug or probiotic intended for promoting juvenile growth in the case of malnutrition characterized by protein-deficiency.

11. The composition according to claim 1, for use as a drug or probiotic intended to promote juvenile growth in the case of malnutrition characterized by an IGF-1 serum level less than the normal level.

12. The composition according to claim 1, wherein the bacterium is a Lactobacillus plantarum.

13. The composition according to claim 12, wherein the bacterium is a Lactobacillus plantarum G821 deposited at the “Collection Nationale de Culture de Microorganismes” (Institut Pasteur) under the registration number CNCM I-4979.

14. The composition according to claim 1, containing from 10⁵ to 10¹²-CFUs of Lactobacillus positively reacting to the test, per gram of composition.

15. A probiotic treatment method for promoting juvenile growth in the case of malnutrition, comprising the administration to a patient in need thereof, of a composition according to claim 1.

16. A method for screening bacteria capable of promoting juvenile growth in the case of malnutrition of the mammals, notably humans, productive animals, pet animals and sport animals, wherein the bacterial strain is subject to the following test:
a batch of embryos of axenic Drosophila melanogaster parents is available;
these embryos are placed in at least 2 groups on a nutritional medium comprising a nutrient medium for Drosophilas deficient in yeast, one of the groups of embryos is further inoculated with a suspension of the bacterium to be tested, which forms the group of monoxenic individuals; the other group forms the axenic group,
the breeding is conducted at a suitable temperature,
at the end of a suitable breeding period, Drosophila larvae of each group (axenic larvae and monoxenic larvae) are recovered,
the average of the length of the larvae of each of the groups is determined and the obtained averages are compared;
the tested bacterial strain is considered as reacting positively to the test if the average of the length of the larvae of the monoxenic group is greater than the average of the length of the larvae of the axenic group with a value of p of less than or equal to 0.001, in the Mann-Whitney test, conducted on the whole of the set of data.

17. A method for screening bacteria capable of promoting juvenile growth in the case of malnutrition, wherein the bacterial strain is subject to the following test:
juveniles are available issued from two lines issued from a same mouse strain, i.e. a line of axenic mice and a line of monoxenic mice,
they are raised in a protein-deficient and also preferably lipid-deficient nutritional diet;
at the end of a suitable breeding period, the average of the values of one or several parameters of each group is determined, these parameters being related to the IGF-1 serum level or to the growth,
the lactic bacterial strain is considered as positively reacting to the test if the average IGF-1 level of the monoxenic group is greater than the average level of the axenic group with a value of p of less than or equal to 0.05 in the Tukey statistical test.

18. A composition, a pure culture or an isolate comprising the Lactobacillus plantarum G821 bacterium deposited at the “Collection Nationale de Culture de Microorganismes” (Institut Pasteur) under the registration number CNCM I-4979.
